(19) United States
(12) Patent Application Publication
(10) Pub. No.: US 2022/0362322 A1
(43) Pub. Date: Nov. 17, 2022

(54) COMPOSITION FOR INHIBITING TUMOR PROLIFERATION COMPRISING SALVIA PLEBEIA EXTRACT AS EFFECTIVE COMPONENT

(71) Applicant: KOREA INSTITUTE OF ORIENTAL MEDICINE, Daejeon (KR)

(72) Inventors: Hwan Suck CHUNG, Daegu (KR); Jang Gi CHOI, Daegu (KR); Young Soo KIM, Gyeonggi-do (KR); Ji Hye KIM, Daegu (KR); Wei LI, Daegu (KR); Taein KIM, Daegu (KR)

(21) Appl. No.: 17/773,243

(22) PCT Filed: Sep. 29, 2020
(86) PCT No.: PCT/KR2020/013292

§ 371 Date: Apr. 29, 2022

(30) Foreign Application Priority Data
Oct. 31, 2019 (KR) 10-2019-0137681

(51) Int. Cl. A61K 36/537; A61K 45/06; A61P 35/00

(52) U.S. Cl. CPC A61K 36/537; A61K 45/06; A61P 35/00

(57) ABSTRACT

A composition according to an embodiment of the present disclosure can be used for inhibiting tumor proliferation and includes a Salvia plebeia extract as an effective component. It was found that, as a result of binding to PD-L1 present on a surface of cancer cell, Salvia plebeia extract blocks the interaction between cancer cell and PD-1 present on a surface of immune T cell to activate T cells and exhibit an inhibitory effect on tumor proliferation. It was also found that, according to co-culture of cells of colon cancer, breast cancer, or lung cancer with T cells, the group treated with Salvia plebeia extract shows higher effect of causing cancer cell death.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2020/013292, filed Sep. 29, 2020, which claims priority to the benefit of Korean Patent Application No. 10-2019-0137681 filed in the Korean Intellectual Property Office on Oct. 31, 2019, the entire contents of which are incorporated herein by reference.

### BACKGROUND

### 1. Technical Field

The present invention relates to a composition for inhibiting tumor proliferation comprising Salvia plebeia extract as an effective component.

### 2. Background Art

As a common anti-cancer therapy for overcoming cancer, tumorectomy is known. For reducing tumor size before surgery, causing death of cancer cells which still remain after surgery, or preventing recurrence of cancer, radiation therapy and chemotherapy are also employed in combination with surgery. Radiation therapy and chemotherapy, which are called as the first-generation anti-cancer agent, are methods of inducing cancer cell death by interfering the pathway of cancer cells to unlimited division and proliferation. However, as the radiation therapy requires high energy beam radiation, a problem occurs in that not only the DNA damage of cancer cell but also death of normal cell is caused.

Moreover, even in case of the chemotherapy involving administration of toxic chemicals for inhibiting cell division process, division of normal cells is also inhibited instead of specific inhibition of the cancer cell division. As such, there is a problem of having serious adverse effects like reduced number of white blood cells, hair loss, and the like. To overcome those problems, a target anti-cancer agent, which is the second-generation anti-cancer agent for selectively attacking cancer cells instead of normal cells, has been developed. It is accordingly expected that the adverse effects of the first-generation anti-cancer agent can be minimized.

Meanwhile, the anti-cancer agent is characterized in that it exhibits its therapeutic effect by acting only on a cancer-causing protein to selectively inhibit the cancer cells.

However, the cancer-causing protein and the protein showing the therapeutic effect are different from each other depending on the type of cancer. Thus, it is necessary to use an anti-cancer agent that is a match with the specific type of a target protein. Moreover, based on a mechanism of acquiring resistance to a target anti-cancer agent, cancer cells have the “immune cell evasion property” for causing mutation so that the cancer cells themselves do not become a target of the target anti-cancer agent. Accordingly, there may be a case in which the target anti-cancer agent is not able to recognize the cancer cells.

As such, the third-generation immune anti-cancer agent is currently developed by which problems associated with the adverse effects and resistance caused by anti-cancer therapy are reduced and remembering and attacking the cancer cells by immune cells are continuously achieved even after the termination of drug administration.

The immune anti-cancer therapy is a treatment method of activating the immune system of a human body so as to enhance the autoimmunity and enable immune cells to attack cancer cells. As such, by focusing on the activity of immune cells, the immune anti-cancer agent is to awaken the “potential (of immune cells) for attacking cancer cells.”

The immune anti-cancer agent can be categorized into an immune checkpoint inhibitor, an immune cell therapeutic agent, a therapeutic antibody, and an anti-cancer vaccine. Immune checkpoint inhibitor is an anti-cancer agent for attacking cancer cells based on blockage of the activity of immune checkpoint protein and activation of T cells. An antibody recognizing CTLA-4, PD-1, or PD-L1 is mainly used as an immune checkpoint inhibitor. As an anti-cancer agent for enhancing the cell immunity, mention can be made of an NK cell therapeutic agent, a T cell therapeutic agent, a CAR-T cell therapeutic agent, and the like. Therapeutic antibody is to attack cancer cells as a drug compound is released upon binding of an antibody drug conjugate to cancer cells. Furthermore, anti-cancer vaccine is an immunotherapy for attacking cancer cells according to activation of the immunity in human body, which is achieved by administering, to a cancer patient, a tumor-specific antigen presents in cancer cells or a protein/peptide molecule for enhancing the overall immune response.

Salvia plebeia is Salvia plebeia R. Br. plant belonging to Labiatae, and it is also called morel cabbage, chongwacho, mamacho, gwadongchong, sooyangee, or the like. It is known to contain, in addition to flavonoids, homoplantaginin, hispidulin, eupafolin, and eupafolin-7-glucoside, components like phenolic material, essential oil, and saponin.

As a study related to Salvia plebeia, a composition for preventing or treating respiratory inflammatory disease comprising specific crude extract or purified fraction isolated from Salvia plebeia as an effective component is described in Korean Patent Publication No. 2016-0146007. In addition, in Korean Patent Publication No. 2011-0078672, a cosmetic composition comprising Salvia plebeia extract as an effective component is disclosed. However, so far there is no disclosure of a composition for inhibiting tumor proliferation comprising Salvia plebeia extract as an effective component as it is described in the present invention.

### SUMMARY

The present invention is devised under the circumstances that are described above, and the present invention provides a composition for inhibiting tumor proliferation comprising Salvia plebeia extract as an effective component. It was found that, as a result of binding to PD-L1 present on a surface of cancer cell or PD-1 present on a surface of immune T cell, Salvia plebeia extract blocks the interaction between cancer cell and PD-1 present on a surface of T cell to activate T cells, thus exhibiting an inhibitory effect on tumor proliferation. It was also found that, according to co-culture of cells of colon cancer, breast cancer, or lung cancer with immune T cells, the group treated with Salvia plebeia extract showed higher effect of causing cancer cell death, and the present invention is completed accordingly.

To achieve the purpose described above, the present invention provides a functional health food composition for immune checkpoint inhibition comprising Salvia plebeia extract as an effective component.

The present invention further provides a pharmaceutical composition for preventing or treating a disorder related to immune checkpoint inhibition comprising Salvia plebeia extract as an effective component.

The present invention further provides an anti-tumor pharmaceutical composition comprising anti-cancer active agent and Salvia plebeia extract as an effective component.

The present invention still further provides anti-cancer adjuvant comprising Salvia plebeia extract as an effective component.

The present invention is devised under the circumstances that are described in the above, and the present invention relates to a composition for inhibiting tumor proliferation comprising Salvia plebeia extract as an effective component. It was found that, as a result of blocking the interaction (i.e., binding) between PD-L1 present on a surface cancer cell and PD-1 present on a surface of immune T cell, Salvia plebeia extract as an effective component of the present invention activates immune T cells and thus exhibits an inhibitory effect on tumor proliferation. It was also found that, according to co-culture of cells of colon cancer, breast cancer, or lung cancer with T cells, the group treated with Salvia plebeia extract shows higher effect of causing cancer cell death.

### BRIEF DESCRIPTION OF THE DRAWINGS

FIG. 1 shows the concentration-dependent inhibition of PD-1/PD-L1 binding when PD-1 binding is allowed to occur after treating a plate, which has been previously coated with PD-L1, with 0.1 to 100 μg/ml anti-PD-L1. ** and *** indicate that, compared to the group without anti-PD-L1 treatment, the group treated with 0.1 to 100 μg/ml anti-PD-L1 has a statistically significant decrease in the PD-1/PD-L1 binding (**; p<0.01, and ***; p<0.001).

FIG. 2 shows the concentration-dependent inhibition of PD-1/PD-L1 binding when PD-1 binding is allowed to occur after treating a plate, which has been previously coated with PD-L1, with 10 to 500 μg/ml Salvia plebeia extract. *** indicates that, compared to the group without any Salvia plebeia extract treatment, the group treated with Salvia plebeia extract has a statistically significant decrease in the PD-1/PD-L1 binding (p<0.001).

FIG. 3 shows the result indicating that T cell-mediated death of colon cancer cells is enhanced by Salvia plebeia extract. (A) of FIG. 3 is the result of FACS for analyzing hPD-L1 expression in mouse colon cancer cells MC38 (CON) or MC38 cells in which mPD-L1 is replaced by hPD-L1 (hPD-L1-MC38). (B) of FIG. 3 is the result of determining the death of hPD-L1-MC38 cancer cells in Salvia plebeia extract (SPE) treatment group when hPD-L1-MC38 cells as cancer cells and spleen cells containing T cell (SP) are co-cultured. *** indicates that, compared to the control group (CON), the group treated with SPE has a statistically significant decrease in cancer cell viability (p<0.001). (C) of FIG. 3 is a photographic image showing the induced death of the colon cancer cells (MC38) in the group treated with 25 μg/ml Salvia plebeia extract.

FIG. 4 shows the death of cancer cells in the Salvia plebeia extract (SPE) treatment group when human colon cancer cells (HCT116) are co-cultured with T cells. (A) of FIG. 4 is the result of measuring the LDH activity of dead cancer cells that are present in co-culture supernatant, in which *** indicates that, compared to CON, the group treated with Salvia plebeia extract has a statistically significant increase in LDH activity (p<0.001).

(B) of FIG. 4 is the result of measuring the amount of cancer cells of(A) of FIG. 4, in which *, **, and *** indicate that, compared to CON, the group treated with PD-L1 Ab (antibody) or Salvia plebeia extract exhibits a statistically significant increase in cancer cell death (*; p<0.05, **; p<0.01, and ***; p<0.001).

FIG. 5 shows the result of determining, when breast cancer cells (MCF-7) and T cells are co-cultured, the viability of cancer cells after the treatment with Salvia plebeia extract (SPE). (A) of FIG. 5 shows the result after co-culture of the breast cancer cells (MCF-7) and T cells at a ratio of 1:2, and (B) of FIG. 5 shows the result after co-culture of the breast cancer cells (MCF-7) and T cells at a ratio of 1:5. ** and *** indicate that, compared to the group without any treatment with Salvia plebeia extract, the group treated with Salvia plebeia extract has a statistically significant decrease in cancer cell viability (**; p<0.01, and ***; p<0.001).

FIG. 6 shows the result of determining, when lung cancer cells (A549) and T cells are co-cultured, the viability of cancer cells after the treatment with Salvia plebeia extract (SPE). (A) of FIG. 6 shows the result after co-culture of the lung cancer cells (A549) and

T cells at a ratio of 1:2, and (B) of FIG. 6 shows the result after co-culture of the lung cancer cells (A549) and T cells at a ratio of 1:5. ** and *** indicate that, compared to the group without any treatment with Salvia plebeia extract, the group treated with Salvia plebeia extract has a statistically significant decrease in cancer cell viability (**; p<0.01, and ***; p<0.001).

FIG. 7 shows the result of determining, after administering the Salvia plebeia extract (SPE) of the present invention, the body weight (B) and the reduction in tumor size (C) according to the passage of time (A), in which the test was made with an animal model of colon cancer. *** indicates that, compared to the group with induced cancer (i.e., vehicle), the group administered with Salvia plebeia extract of the present invention or the positive control has a statistically significant decrease in tumor size (p<0.001).

### DETAILED DESCRIPTION

The present invention relates to a functional health food composition for immune checkpoint inhibition comprising Salvia plebeia extract as an effective component. The Salvia plebeia extract can be produced by a method including the following steps:

(1) carrying out extraction by adding an extraction solvent to Salvia plebeia;

(2) filtering the extract of the step (1); and (3) concentrating under reduced pressure the filtered extract of the step (2) followed by drying to produce extract, but the method is not limited thereto.

The extraction solvent of the above step (1) is preferably selected from water, C₁-C₄ lower alcohol, and a mixture thereof. It is more preferably C₁-C₄ lower alcohol and even more preferably 70% (v/v) ethanol, but not limited thereto.

With regard to the production method, any kind of common methods that are generally known as extraction method in the pertinent art, e.g., filtration, hot water extraction, impregnation extraction, extraction by reflux condensation, and ultrasonic extraction, can be used. It is preferable that the extraction is carried out by adding an extraction solvent in an amount of 1 to 20 times the weight of Salvia plebeia. More preferably, the extraction solvent is added in an amount of 5 to 15 times the weight of Salvia plebeia. The extraction temperature is preferably between 4° C. and 50° C., but it is not limited thereto. Furthermore, the extraction time is preferably between 0.5 hour and 10 hours, and more preferably between 0.5 hour and 5 hours, but it is not limited thereto. It is preferable that the concentration of the step (3) in the above method uses a vacuum rotary condenser or a vacuum rotary evaporator, but it is not limited thereto. Furthermore, the drying is preferably carried out by drying under reduced pressure, drying under vacuum, drying under boiling, spray drying, or freeze-drying, but it is not limited thereto.

The Salvia plebeia extract is characterized in that it has PD-L1 or PD-1 as a target. The cancer cell is preferably a cancer cell originating from any cancer selected from colon cancer, breast cancer, lung cancer, melanoma, liver cancer, stomach cancer, colorectal cancer, skin cancer, bladder cancer, prostate cancer, ovary cancer, cervical cancer, thyroid cancer, kidney cancer, fibrosarcoma, and leukemia, but it is not limited thereto.

Salvia plebeia extract may be directly added to the functional health food composition of the present invention, or it may be used with other food product or food ingredient, and it can be suitably used according to a common method. Type of the functional health food composition is not particularly limited. Examples of the food to which the Salvia plebeia extract can be added include meat, sausage, bread, chocolate, candies, snacks, biscuits, pizza, ramen, other noodles, gums, dairy products including ice cream, various kinds of soup, beverage, tea, drink, alcohol beverage, and vitamin complex, and all functional health food products in general sense are included therein. Like common beverages, health drink containing the composition of the present invention may comprise various flavoring agents and natural carbohydrates as an additional component. Examples of the natural carbohydrates include monosaccharides like glucose and fructose, disaccharides like maltose and sucrose, polysaccharides like dextrin and cyclodextrin, and sugar alcohols like xylitol, sorbitol, and erythritol. As a sweetening agent, natural sweetening agent like thaumatin and stevia extract and synthetic sweetening agent like saccharine and aspartame can be used. Amount of the natural carbohydrates is generally about 0.01 g to 0.04 g, and preferably about 0.02 g to 0.03 g relative to 100 g of the composition of the present invention.

The functional health food composition of the present invention may further comprise, in addition to the aforementioned effective component, various nutritional supplements, a vitamin, an electrolyte, a flavor, a coloring agent, pectinic acid and a salt thereof, alginic acid and a salt thereof, an organic acid, a protective colloidal thickening agent, a pH adjusting agent, a stabilizer, a preservative, glycerin, alcohol, and a carbonating agent used for carbonated drink. Other than those, fruit juice or fruit pulp for producing vegetable drink may be additionally comprised. Those ingredients may be used either singly or in combination thereof. Amount of the above various additives is not critical, but it is generally selected from a range of about 0.01 to 2 parts by weight relative to 100 parts by weight of the composition of the present invention.

The present invention further relates to a pharmaceutical composition for preventing or treating a disorder related to immune checkpoint inhibition comprising Salvia plebeia extract as an effective component.

The disorder related to immune checkpoint inhibition is preferably cancer, infection, sepsis, or immune aging. It is more preferably cancer, but not limited thereto. The cancer is the same as defined in the above.

The present invention further relates to an anti-tumor pharmaceutical composition comprising an anti-cancer active agent and Salvia plebeia extract as an effective component.

The anti-cancer active agent is preferably an anti-cancer agent or an immune checkpoint inhibitor, but it is not limited thereto. The anti-cancer agent is preferably one or more selected from actinomycin D, bleomycin sulfate, daunomycin, daunorubicin, doxorubicin, epirubicin, idarubicin, mitomycin, mitomycin-C, mithramycin, irinotecan, camptothecin, novobiocin, epirubicin, dactinomycin, amsacrine, teniposide, etoposide, cisplatin, carboplatin, oxaliplatin, paclitaxel, docetaxel, gefitinib, erlotinib, and afatinib, and the immune checkpoint inhibitor is preferably anti PD-L1 antibody, anti PD-1 antibody, anti CD-80 antibody, or anti CTLA-4 antibody, but it is not limited thereto.

The pharmaceutical composition of the present invention may be in various types of preparation such as oral preparation or parenteral preparation. In case of producing a preparation, the preparation is made by using a diluent or a vehicle such as filler, bulking agent, binding agent, moisturizing agent, disintegrating agent, or surfactant that are commonly used for producing a preparation. Examples of the solid preparation for oral administration include a tablet, a pill, a powder, a granule, and a capsule. The solid preparation is produced by mixing at least one compound with one or more vehicles such as starch, calcium carbonate, sucrose, lactose, or gelatin. Furthermore, other than simple vehicles, a lubricating agent such as magnesium stearate or talc is also used. As for the liquid preparation for oral administration, a suspension, a solution preparation for internal use, an emulsion, a syrup preparation, or the like can be mentioned. Other than water or liquid paraffin commonly used as a simple diluent, various kinds of a vehicle such as moisturizing agent, sweetening agent, aromatic agent, or preservatives may be included.

Examples of a preparation for parenteral administration include a sterilized aqueous solution, a non-soluble preparation, a suspension preparation, an emulsion preparation, a freeze-dried preparation, and a suppository preparation. As a water insoluble solvent or a suspending preparation, propylene glycol, polyethylene glycol, or vegetable oil such as olive oil, and injectable ester such as ethylolate can be used. As a base for a suppository preparation, witepsol, macrogol, tween 61, cacao fat, laurin fat, glycerol, gelatin, or the like can be used.

The pharmaceutical composition of the present invention can be administered either orally or parenterally. In case of parenteral administration, it is preferable to choose external application on skin, or intraperitoneal, rectal, intravenous, muscular, subcutaneous, endometrium injection, or intracerebroventricular injection.

The pharmaceutical composition of the present invention is administered in a pharmaceutically effective amount. As described herein, the expression “pharmaceutically effective amount” means an amount sufficient for treating a disorder at reasonable benefit-risk ratio that can be applied for a medical treatment. The effective dose level may be determined based on a type or severeness of a disorder of a patient, activity of a pharmaceutical, sensitivity to a pharmaceutical, administration period, administration route, excretion ratio, time period for therapy, elements including a pharmaceutical used in combination, and other elements that are well known in the medical field. The composition of the present invention can be administered as a separate therapeutic agent, or it can be used in combination with other therapeutic agent. It can be administered in order or simultaneously with a conventional therapeutic agent. It can be also administered as single-dose or multi-dose. It is important to administer an amount which allows obtainment of the maximum effect with minimum dose while considering all of the aforementioned elements without having any side effect, and the dosage can be easily determined by a person skilled in the pertinent art.

The dosage of the composition of the present invention may vary depending on body weight, age, sex, health state, diet of a patient, administration period, administration method, excretion rate, and severeness of disorder. However, the daily dosage is, in terms of the amount of Salvia plebeia extract, 0.01 to 2,000 mg/kg, preferably 30 to 500 mg/kg, and more preferably 50 to 300 mg/kg, and it can be administered 1 to 6 times per day. The pharmaceutical composition of the present invention may be used either singly, or in combination with other treatment methods including surgery, radiation therapy, hormone therapy, chemotherapy, antibody therapy, and a method of using biological response modifier.

The present invention still further relates to an anti-cancer adjuvant comprising Salvia plebeia extract as an effective component.

The anti-cancer adjuvant may comprise, in addition to the Salvia plebeia extract, one or more types of an effective component which exhibits similar or even identical effect to the extract. For clinical administration, the anti-cancer adjuvant may be administered either orally or parenterally. In case of parenteral administration, it may be administered via intraperitoneal injection, intrarectal injection, subcutaneous injection, intravenous injection, muscular injection, endometrium injection, intracerebroventricular injection, or intrathoracic injection, and it can be used in the form of a common pharmaceutical preparation.

The anti-cancer adjuvant may be used either singly, or in combination with other methods including surgery, radiation therapy, hormone therapy, chemotherapy, and a method of using biological response modifier. The dosage of the anti-cancer adjuvant is about 0.0001 to 100 mg/kg, and preferably 0.001 to 10 mg/kg per day, and the administration can be made once a day, or several times a day with divided portion. The dosage is within a broad range depending on body weight, age, sex, health state, and diet of a patient, administration period and administration method, excretion rate, and severeness of disorder. For clinical administration, the anti-cancer adjuvant of the present invention may be administered parenterally in various preparation types, and in case of producing a preparation, the preparation can be made by using a diluent or a vehicle like a filling agent, a bulking agent, a binding agent, a wetting agent, a disintegrating agent, and a surfactant that are commonly used. Examples of a preparation for parenteral administration include a sterilized aqueous solution, a non-soluble preparation, a suspension preparation, an emulsion preparation, a freeze-dried preparation, and a suppository preparation. As a water insoluble solvent or a suspending preparation, propylene glycol, polyethylene glycol, or vegetable oil such as olive oil, and injectable ester such as ethylolate can be used. As a base for a suppository preparation, witepsol, macrogol, tween 61, cacao fat, laurin fat, glycerol, gelatin, or the like can be used.

Hereinbelow, the present invention is explained in greater detail in view of the Examples. However, the following Examples are given only for specific explanation of the present invention and it would be evident to a person who has common knowledge in the pertinent art that the scope of the present invention is not limited by them.

### EXAMPLE 1

### Preparation of Salvia plebeia Extract

After drying 10 g of Salvia plebeia, 70% (v/v) ethanol (100 ml) was added thereto as a solvent. Reflux extraction of the mixture was carried out three times for 2 hours followed by concentration under reduced pressure to prepare Salvia plebeia extract.

### EXAMPLE 2

### Competitive ELISA Analysis of Interaction between Ethanol Extract of Salvia plebeia and PD-L1

By utilizing competitive ELISA analysis, concentration-dependent binding of the ethanol extract of Salvia plebeia to PD-L1 was determined to examine the blockage of the interaction with PD-1.

The competitive ELISA analysis was carried out according to the manufacturer's protocol using the PD-1/PD-L1 inhibitor screening kit. Recombinant human PD-L1 (1 μg/ml) dissolved in PBS was coated overnight on a 96-well plate. The 96-well plate was then washed with PBS containing 0.1% Tween (PBS-T) and blocked at room temperature for 1 hour with PBS containing 2% (w/v) BSA followed by washing again. After that, 0.5 μg/ml biotinylated hPD-1 (50 μl) was added to each well, and the plate was incubated for 2 hours at room temperature. After washing 3 times with PBS-T, 50 μl of streptavidin conjugated with HRP (0.2 μg/ml) were added to each well, and the plate was incubated for 1 hour. After the incubation, the plate was washed 3 times with 0.1% PBS-T, and relative chemical luminescence was measured by using SpectraMax L luminometer.

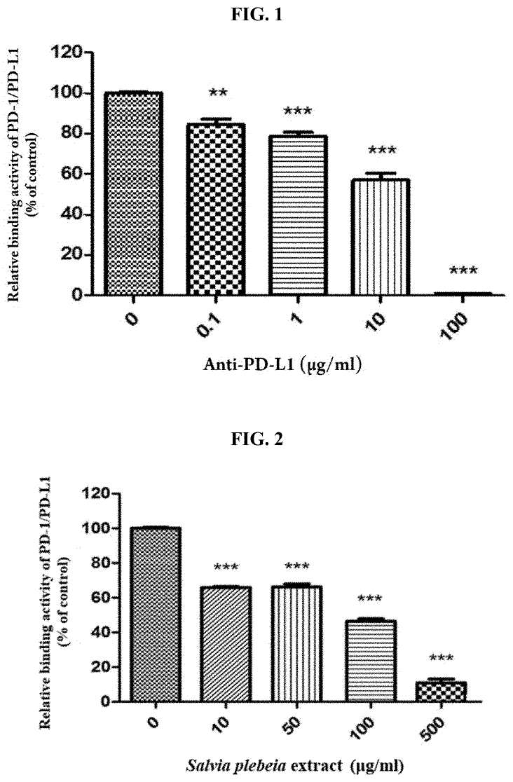

As the result is shown in FIG. 1, it was found that PD-1/PD-L1 binding was inhibited in concentration dependent manner, and Salvia plebeia extract inhibits, with statistical significance, PD-1/PD-L1 binding in concentration dependent manner (FIG. 2). Based on this result, it was recognized that T cell activation is promoted via immune checkpoint inhibition, and thus the effect of inhibiting tumor proliferation against cancer cells is exhibited.

### EXAMPLE 3

### Determination of Enhanced T Cell-Mediated Death of Colon Cancer Cells by Salvia plebeia Extract

### (1) Flow Cytometry

To determine the expression of hPD-L1 on surface of MC38 cells, flow cytometry was carried out. Specifically, to detect the surface expression of hPD-L1 (CD274) in MC38 cells, cells were suspended in phosphate-buffered physiological saline (PBS) with 0.2% bovine serum albumin. Then, the cells were treated on ice with ePC-conjugated anti-human PD-L1 antibody or APC-conjugated mouse IgG1 as an isotype control group followed by incubation for 30 minutes. Thereafter, the cells were washed with PBS and examined using CytoFLEX flow cytometer (Beckman, Brea, Calif., USA). The obtained data were analyzed using FlowJo Software version v10.

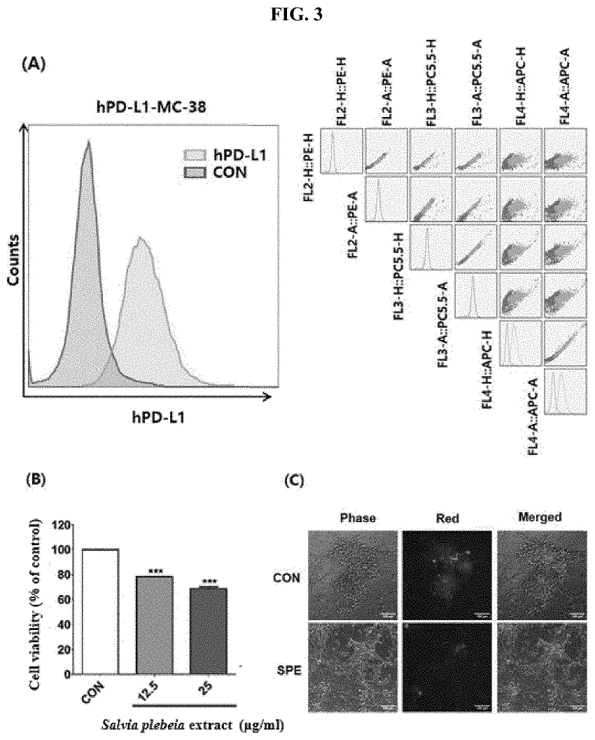

As the result is shown in (A) of FIG. 3, it was found that normal expression of hPD-L1 is obtained.

### (2) CCK Assay

Cell viability was measured by using CCK assay. On a 96 well plate, cells were spread in an amount of 1×10⁴ cells/well and cultured overnight. After that, Salvia plebeia extract was added at each different concentration. CCK solution (10 μl) was then added and the plate was incubated for 2 hours at 37° C. Thereafter, color intensity was measured at 450 nm by using a microplate reader.

As the result is shown in (B) of FIG. 3 and (C) of FIG. 3, it was found that cell death is caused by a treatment with Salvia plebeia extract.

### (3) Measurement of LDH Activity and Determination of Cancer Cell Death by Staining

LDH activity and cancer cell death in Salvia plebeia extract (SPE) treatment group were determined after co-culture of human colon cancer cells (HCT116) and T cells. Specifically, colon cancer cells (HCT116) which have been treated with 10 ng/ml interferon gamma were spread on a 96 well plate in an amount of 5×10⁴ cells/well and cultured for 18 hours to have adhesion on the plate bottom. After that, T cells having overexpressed PD-1 were aliquoted, in an amount of 1×10⁵ cells/well, to the 96 well plate adhered with colon cancer cells, Salvia plebeia extract (12.5 or 25 μg/ml) was added, and co-culture was carried out for 72 hours. After that, the top part of the culture broth was aliquoted to a fresh 96 well plate and reagents for LDH assay were added to have a reaction. Once the reaction is over, a solution for terminating the reaction was added, and the absorbance at 540 nm was measured for each sample.

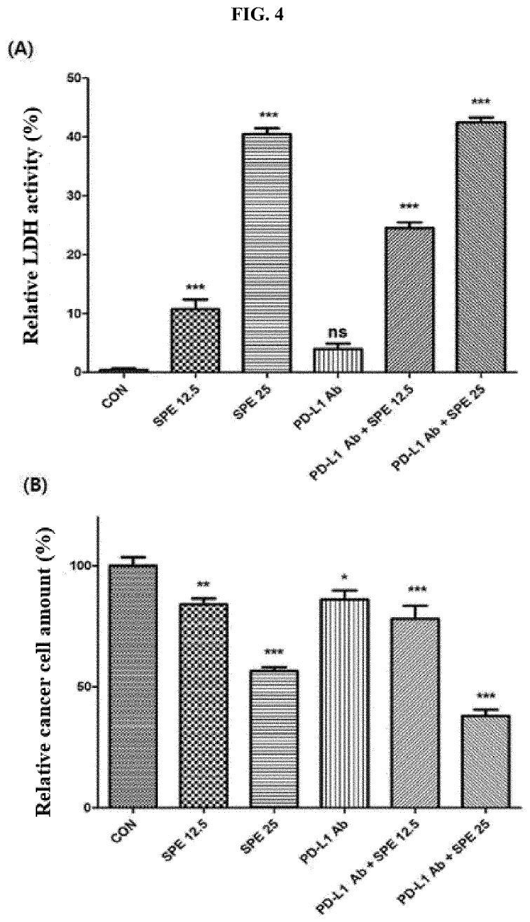

As the result is shown in FIG. 4, it was found that, when co-culture is carried out following the treatment with Salvia plebeia extract, the LDH activity is enhanced to cause the death of cancer cells.

### EXAMPLE 4

### Determination of Enhanced Death of Breast Cancer Cells and Lung Cancer Cells caused by Salvia plebeia Extract Treatment and T Cell co-culture

### (1) Determination of Effect of Causing Breast Cancer Cell Death

Cancer cell death according to co-culture with breast cancer cells (MCF-7) and T cells and also a treatment with Salvia plebeia extract (SPE) was determined. Co-culture with breast cancer cells (MCF-7) and T cells was carried out at a ratio of 1:2 or 1:5.

Specifically, breast cancer cells (MCF-7) which have been treated with 10 ng/ml interferon gamma were spread on a 96 well plate in an amount of 5×10⁴ cells/well and cultured for 18 hours to have adhesion on the plate bottom. After that, T cells having overexpressed PD-1 were aliquoted, in an amount of 1×10⁵ cells/well for co-culture with a ratio of 1:2 or 2.5×10⁵ cells/well for co-culture with a ratio of 1:5, to the 96 well plate adhered with breast cancer cells, Salvia plebeia extract (12.5, 25, 50 or 100 μg/ml) was added, and co-culture was carried out for 72 hours. After that, CCK solution (100 was added followed by incubation for 2 hours at 37° C. Absorbance at 450 nm was then measured for each sample using microplate.

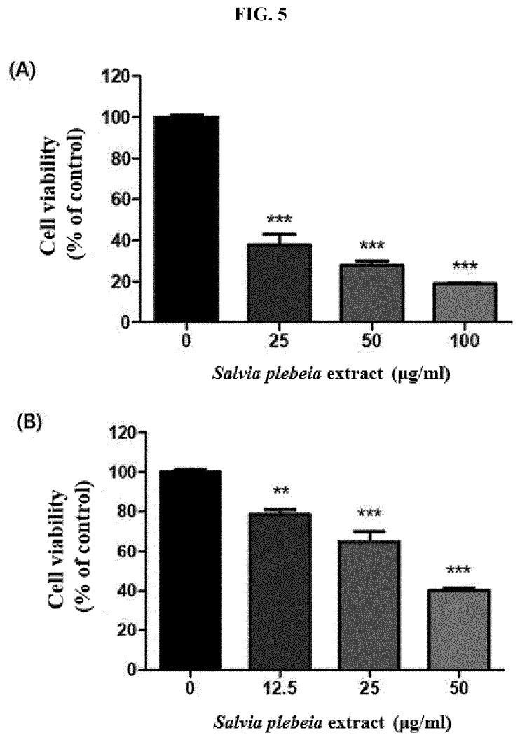

As the result is shown in FIG. 5, it was found that, when co-culture is carried out following the treatment with Salvia plebeia extract, the viability of breast cancer cells has decreased with statistical significance.

### (2) Determination of Effect of Causing Lung Cancer Cell Death

Cancer cell death according to co-culture with lung cancer cells (A549) and T cells and also a treatment with Salvia plebeia extract (SPE) was determined. Co-culture with lung cancer cells (A549) and T cells was carried out at a ratio of 1:2 or 1:5.

Specifically, lung cancer cells (A549) which have been treated with 10 ng/ml interferon gamma were spread on a 96 well plate in an amount of 5×10⁴ cells/well and cultured for 18 hours to have adhesion on the plate bottom. After that, T cells having overexpressed PD-1 were aliquoted, in an amount of 1×10⁵ cells/well for co-culture with a ratio of 1:2 or 2.5×10⁵ cells/well for co-culture with a ratio of 1:5, to the 96 well plate adhered with lung cancer cells, Salvia plebeia extract (12.5, 25, 50 or 100 μg/ml) was added, and co-culture was carried out for 72 hours. After that, CCK solution (10 μl) was added followed by incubation for 2 hours at 37° C. Absorbance at 450 nm was then measured for each sample using microplate.

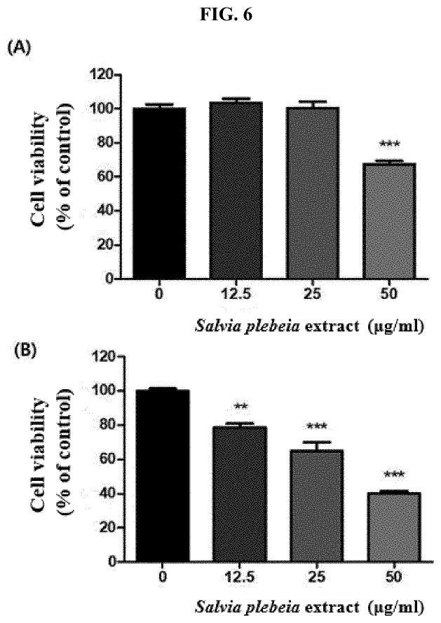

As the result is shown in FIG. 6, it was found that, when co-culture is carried out following the treatment with Salvia plebeia extract, the viability of lung cancer cells has decreased with statistical significance.

### EXAMPLE 5

### Determination of Effect of Reducing Tumor Size by Administration of Salvia plebeia Extract in which Determination is made using Animal Model of Colon Cancer

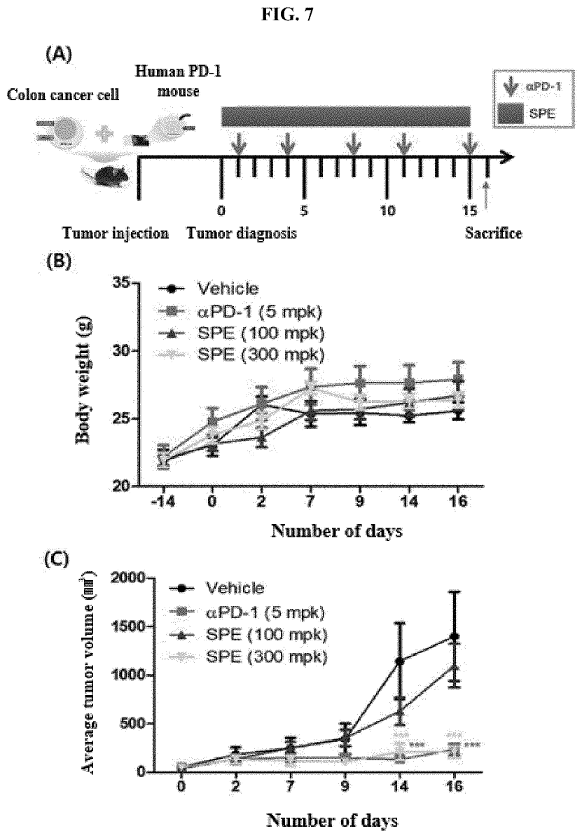

A mouse (C57BL/6J) in which mPD-1 has been removed and replaced by hPD-1 was used for the test, i.e., six animals for each group. MC38 cells (mouse colon cancer cells) obtained by removing mouse PD-L1 (mPD-L1) and replacing it with human PD-1 (hPD-L1) were injected to the animal. Starting from the 14^{th} day after the injection, aPD-1 (positive control) or Salvia plebeia extract (100 or 300 mg/kg) was administered with a time interval shown in FIG. 7. Body weight of the animal and tumor size were then measured.

As the result is shown in FIG. 7, it was found that the tumor size has been reduced with statistical significance in the Salvia plebeia extract administration group of the present invention and the positive control group when compared to the group induced to have cancer (i.e., vehicle), while there is no clear change in the mouse body weight.

1-12. (canceled)

13. A method for immune checkpoint inhibition, the method comprising administering a composition comprising a Salvia plebeia extract as an effective component to a subject in need thereof.

14. The method of claim 13, wherein the Salvia plebeia extract is produced by using an extraction solvent selected from the group consisting of water, a C1-C4 lower alcohol, and a mixture thereof.

15. The method of claim 13, wherein the Salvia plebeia extract has PD-L1 or PD-1 as a target.

16. The method of claim 15, wherein the PD-L1 is found on cancer cell which is a cell originating from any cancer selected from colon cancer, breast cancer, lung cancer, melanoma, liver cancer, stomach cancer, colorectal cancer, skin cancer, bladder cancer, prostate cancer, ovary cancer, cervical cancer, thyroid cancer, kidney cancer, fibrosarcoma, and leukemia.

17. A method for treating a disorder related to immune checkpoint inhibition, the method comprising administering a composition comprising a Salvia plebeia extract as an effective component to a subject in need thereof.

18. The method of claim 17, wherein the disorder related to immune checkpoint inhibition is at least one of cancer, infection, sepsis, and immune aging.

19. The method of claim 18, wherein the disorder related to immune checkpoint inhibition is the cancer selected from the group consisting of colon cancer, breast cancer, lung cancer, melanoma, liver cancer, stomach cancer, colorectal cancer, skin cancer, bladder cancer, prostate cancer, ovary cancer, cervical cancer, thyroid cancer, kidney cancer, fibrosarcoma, leukemia, and a combination thereof.

20. A method for treating a cancer, the method comprising administering a composition comprising an anti-cancer active agent and a Salvia plebeia extract as an effective component to a subject in need thereof.

21. The method of claim 20, wherein the anti-cancer active agent comprises at least one of an anti-cancer agent and an immune checkpoint inhibitor.

22. The method of claim 21, wherein the anti-cancer active agent comprises the anti-cancer agent selected from the group consisting of actinomycin D, bleomycin sulfate, daunomycin, daunorubicin, doxorubicin, epirubicin, idarubicin, mitomycin, mitomycin-C, mithramycin, irinotecan, camptothecin, novobiocin, epirubicin, dactinomycin, amsacrine, teniposide, etoposide, cisplatin, carboplatin, oxaliplatin, paclitaxel, docetaxel, gefitinib, erlotinib, afatinib, and a combination thereof.

23. The method of claim 21, wherein the anti-cancer active agent comprises the immune checkpoint inhibitor selected from the group consisting of anti PD-L1 antibody, anti PD-1 antibody, anti CD-80 antibody, anti CTLA-4 antibody, and a combination thereof.

24. The method of claim 20, wherein the composition includes the anti-cancer active agent and an anti-cancer adjuvant comprising the Salvia plebeia extract.
